(19) United States
(12) Patent Application Publication
(10) Pub. No.: US 2025/0067726 A1
(43) Pub. Date: Feb. 27, 2025

(54) METHOD FOR ANALYZING PERSONALIZED ANTI-CANCER DRUGS IN CELL CULTURES

(71) Applicant: PreComb Therapeutics AG, Hombrechtikon (CH)

(72) Inventors: Jens Kelm, Uster (CH); Peter Steiner, Altendorf (CH); Olivier Mauti, Zürich (CH)

(21) Appl. No.: 18/726,740

(22) PCT Filed: Dec. 28, 2022
(86) PCT No.: PCT/EP2022/087938

§ 371 Date: Jul. 3, 2024

(30) Foreign Application Priority Data
Jan. 4, 2022 (DE) 10 2022 100 146.6

(51) Int. Cl. G01N 33/50

(52) U.S. Cl. CPC G01N 33/5011; G01N 33/5082

(57) ABSTRACT

The present invention relates to improved methods and systems to analyze physiological effects as a response of cells as obtained to the exposure of a drug compound or combinations thereof. The methods of the present invention offer the particular advantage of providing more relevant results with respect to the in vivo situation, are time-and cost effective, and suitable for automatization.

The present invention relates to improved methods and systems to analyze physiological effects as a response of cells as obtained to the exposure of a drug compound or combinations thereof. The methods of the present invention offer the particular advantage of providing more relevant results with respect to the in vivo situation, are time-and cost effective, and suitable for automatization.

### BACKGROUND OF THE INVENTION

Drugs in development for therapeutic purposes are generally screened for efficacy in a conventional screening system, where drugs from a library are tested in a suitable cell-based assay. Usually, the viability of the cells and/or the cytotoxicity of the candidate drug are investigated. This approach often is done in a so-called high throughput, but still there is a high risk that drugs identified as promising in such approach will later disappoint in the subsequent clinical testing. Conventionally, drugs are combined empirically, by medical practitioners, and tested in patients.

Despite all efforts, 97% of all cancer drugs entering phase I clinical trials fail and cannot be brought to the market, since pre-clinical drug tests do not produce accurate results. Pharma companies spend $14bn on potential new cancer drugs that fail clinical trials.

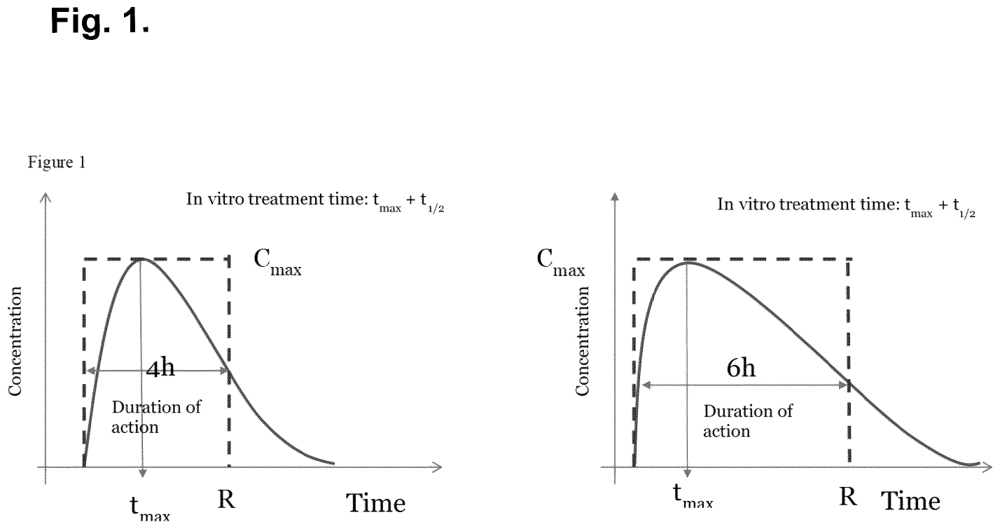

Following the oral administration of drugs, the plasma concentration generally reaches, in principle, a single, well-defined peak (Cmax) at the time of Tmax. The value T½ defines the half-life of the drug, i.e. the time at which half of the drug is eliminated from the system (see FIG. 1). In a cellular test system using a multitude of compounds or drugs to be tested, usually all drugs are applied to the cells at the same time. In tests in cell culture, the drug is immediately available at the maximum concentration level. While this does not reflect the situation in vivo, the situation is at least controllable.

In cultures involving layers of cells or 3D tissue or microtissue cultures, the situation is more complex. For example Wang et al. (in: Wang Y, et al. Modeling Endothelialized Hepatic Tumor Microtissues for Drug Screening. Adv Sci (Weinh). 2020 Sep. 21;7 (21): 2002002. doi: 10.1002/advs.202002002. PMID: 33173735; PMCID: PMC7610277) disclose the development of a 3D endothelialized hepatic tumor microtissue model based on the fusion of multicellular aggregates of human hepatocellular carcinoma cells and human umbilical vein endothelial cells co-cultured in poly (lactic-co-glycolic-acid) based porous microspheres (PLGA PMs). In contrast to the conventional 2D culture, the cells within the PLGA PMs exhibit significantly higher half-maximal inhibitory concentration values against anticancer drugs, including doxorubicin and cisplatin. Furthermore, the feasibility of co-culturing other cell types, such as fibroblasts (L929) and HepG2 cells, was investigated.

WO 2020/242594A1 describes methods and apparatuses that form and grow patient-derived micro-organospheres (PMOS) containing cells originating from a patient, for example, extracted from a small patient biopsy, (e.g., for quick diagnostics to guide therapy), from resected patient tissue, including resected primary tumor or part of a dysfunctional organ (e.g., for high-throughput screening), and/or from already established PDMCs, including patient-derived xenografts (PDX) and organoids (e.g., to generate Micro-Organospheres for high-throughput screening), including personalized therapies. The method comprises combining a dissociated tissue sample and a fluid matrix material to form an unpolymerized mixture; forming a plurality of droplets of the unpolymerized mixture; and polymerizing the droplets to form a plurality of Patient-Derived Micro-Organospheres each having a diameter of between 50 and 500 pm with between 1 and 200 dissociated cells distributed therein.

Cultures involving layers of cells or 3D tissue or microtissue cultures are usually employed in order to mimic the situation in a tissue in vivo to as much as possible. Nevertheless, an increase of the physiologic relevance of in vitro testing of multiple drugs in parallel includes the challenge to adapt to pharmacokinetic properties of each drug in order to further improve drug response prediction. In addition, the more drugs are being tested in parallel, the higher the complexity of individual drug exposure time.

This means that the predictability of early stage microtissue experiments with respect to the future in vivo situation needs to be improved, in order to reduce the risk of failure of drugs or drug combinations that have turned out promising in the pre-clinic.

Therefore, it is an object of the invention to provide new and improved methods and device to analyze the phenotype and/or genotype of cells obtained from tissue samples upon exposure of these cells to drugs or drug combinations, in particular in order to streamline and render more effective patient-specific treatment approaches.

The present invention generally relates to an improved screening of drugs or combinations thereof, in particular patient-specific drugs or combinations thereof, for therapeutic purposes. In particular, the present invention relates to the screening of drugs or combinations that can be used therapeutically in the treatment and/or prevention and the slowing down of the progression of diseases, such as neoplastic or tumorous cell growth in an individual in need thercof.

In a first aspect, the present invention solves the above object by providing a method, particularly an automated method, for determining the effectivity of at least one, in particular personalized, drug or drug combination for the treatment of a patient, said method comprising a) providing at least one culture of a 3D microtissue, such as a microtumor, based on dissociated cells of a tissue sample derived from a patient, b) providing a drug or a combination or panel of drugs to be tested, c) identifying the t½ time period(s) for said drug or combination or panel of drugs of step b), c) contacting the at least one 3D microtissue culture with the drug or combination or panel or group of drugs to be tested identified to have the longest t½ time period(s) (T_{L1}), wherein the end of this time period(s) define(s) the time point for a removal R of the drug or panel or group of drugs to be tested from the culture, f) incubating said at least one 3D microtissue culture, preferably an array of cultures, with the drug or combination or panel or group of drugs to be tested, and, optionally, determining an effect of said drugs and/or combinations thereof on at least one physiological parameter of the at least one 3D microtissue culture, g) subsequent to e), contacting the at least one 3D microtissue culture with the drug or combination or panel or group of drugs to be tested identified to have a shorter t½ time period(s) (T_{L2}) compared to the drug or combination or panel or group of drugs to be tested in step e) at time point R minus T_{L2}, h) incubating the at least one 3D microtissue culture with the drugs or combination or panel or group of drugs to be tested, j) removing the drug or panel or combination or group of drugs to be tested from the culture at timepoint R, and k) determining an effect of said drugs and/or combinations thereof on at least one physiological parameter of the at least one drug or panel or combination or group of drugs on the at least one 3D microtissue culture.

Preferred is a method according to the present invention as herein, optionally further comprising at least one of d) pre-selecting or grouping the combination or panel of drugs according to the t½ time periods as identified in step c); i) repeating steps g) and h) with at least one drug or panel combination or group of drugs to be tested identified to have a shorter t½ value or values (T_{L3}, T_{L4}, . . . ) compared to the drug or panel or group of drugs to be tested in the steps beforehand at time points R minus T_{L3}, R minus T_{L4}, . . . , and/or 1) identifying at least one patient-specific drug or combination or panel or group of drugs based on the effect(s) as determined, and, optionally, further comprising the step of selecting said patient-specific drug or combination or panel or group of drugs as identified.

More preferred is this a method for determining the effectivity of at least one, in particular personalized, drug or drug combination for the treatment of a patient, said method comprising a) providing at least one culture of a 3D microtissue, such as a microtumor, based on dissociated cells of a tissue sample derived from a patient, b) providing a drug or a combination or panel of drugs to be tested, c) identifying the t½ time period(s) for said drug or combination or panel of drugs of step b), d) pre-selecting or grouping the combination or panel of drugs according to the t½ time periods as identified in step c), e) contacting the at least one 3D microtissue culture with the drug or combination or panel or group of drugs to be tested identified to have the longest t½ time period(s) (T_{L1}), wherein the end of this time period(s) define(s) the time point for a removal R of the drug or panel or group of drugs to be tested from the culture, f) incubating said at least one 3D microtissue culture, preferably an array of cultures, with the drug or combination or panel or group of drugs to be tested, and, optionally, determining an effect of said drugs and/or combinations thereof on at least one physiological parameter of the at least one 3D microtissue culture, g) subsequent to e), contacting the at least one 3D microtissue culture with the drug or combination or panel or group of drugs to be tested identified to have a shorter t½ time period(s) (T_{L2}) compared to the drug or combination or panel or group of drugs to be tested in step e) at time point R minus T_{L2}, h) incubating the at least one 3D microtissue culture with the drugs or combination or panel or group of drugs to be tested, i) repeating steps g) and h) with at least one drug or panel combination or group of drugs to be tested identified to have a shorter t½ value or values (T_{L3}, T_{L4}, . . . ) compared to the drug or panel or group of drugs to be tested in the steps beforehand at time points R minus T_{L3}, R minus T_{L4}, . . . , j) removing the drug or panel or combination or group of drugs to be tested from the culture at timepoint R, and k) determining an effect of said drugs and/or combinations thereof on at least one physiological parameter of the at least one drug or panel or combination or group of drugs on the at least one 3D microtissue culture, and 1) identifying at least one patient-specific drug or combination or panel or group of drugs based on the effect(s) as determined, and, optionally, further comprising the step of selecting said patient-specific drug or combination or panel or group of drugs as identified.

Further preferred is the method according to the present invention, wherein the at least one culture of a 3D microtissue is contacted with the drug or combination or panel or group of drugs at the Cₘₐₓ concentration.

The methods of the present invention take into account the pharmacokinetic parameter of a drug or group of drugs or panel of drugs to be tested, i.e. the differences in the t½, Cₘₐₓ or Tₘₐₓ or the AUC of a drug or group of drugs or panel of drugs to be tested are considered when screening for efficient drug candidates and panels for a patient. This is of particular importance in case of combinations of drugs that are then jointly tested with respect to their behavior and effect in a microtissue culture.

Utilizing 3D tissue models allows for extended assay times (7-28 days) in order to assess safety and efficacy and is suitable to incorporate differential pharmacokinetic properties of drugs. The inventors have developed a process to minimize complexity and increase assay robustness of dealing with individual drug exposure times for testing multiple drugs in parallel. The workflow is further designed to be fully compatible with automated liquid handling processes (e.g. in a multiwell plate).

In a second aspect, the present invention solves the above object by providing a method for identifying adverse effects associated with a treatment with a patient-specific drug or combination or panel or group of drugs in a patient, comprising performing the method according to the present invention, further comprising the step of testing and analyzing said patient-specific drug or combination or panel or group of drugs for adverse effects in said patient.

In a third aspect, the present invention solves the above object by providing a method for stratifying a patient with respect to a treatment with a patient-specific drug or combination or panel or group of drugs, comprising performing the method according to the present invention, comprising identifying at least one patient-specific drug or combination or panel or group of drugs based on the effect(s) as determined, and stratifying of said patient based on said patient-specific drug or combination or panel or group of drugs as identified.

In a fourth aspect, the present invention solves the above object by providing a method for monitoring the treatment with a patient-specific drug or combination or panel or group of drugs, comprising performing the method according to the present invention, treating said patient with said at least one patient-specific drug or combination or panel or group of drugs as identified, and repeating the method according to the present invention on a sample of said patient as treated, and optionally adjusting the treatment based on the result(s) of said repeating.

Preferred is the method according to the present invention, wherein said step of identifying and/or selecting includes the use of additional clinical parameters, such as patient specific data and/or treatment history.

In a fifth aspect, the present invention solves the above object by providing a testing system comprising means for performing the methods according to the present invention, comprising a) a unit for culturing an array of 3D microtissues, such as microtumors, based on dissociated cells of a tissue sample derived from the patient as provided, b) a drug testing unit for contacting said array of said 3D microtissues with at least two drugs or combination or panel or group of drugs to be tested in accordance with their respective t½ time period(s) (T_{L1}, and/or T_{L2}, TL₃, . . . ), c) a first analysis unit for determining an effect of said drugs or combination or panel or group of drugs thereof on said array of said 3D microtissues, and d) a second drug testing unit for removing the drug or panel or combination or group of drugs to be tested from the culture at timepoint R.

Preferred is the testing system according to the present invention, further comprising at least one database for collecting and/or storing data selected from the group consisting of t½ time periods, data with respect to the pre-selection and/or grouping of the combination or panel of drugs, physiological parameters, data with respect to the effect(s) of said drugs or combination or panel or group of drugs thereof, data with respect to adverse effects, data with respect to additional clinical parameters, such as patient specific data and/or treatment history, data with respect to the stratification and/or monitoring, and data for automatization of the system, for example for controlling at least one robot.

In a sixth aspect, the present invention solves the above object by providing a computer program comprising means for controlling the testing system according to the present invention, in particular a computer program comprising data for at least one patient-specific drug or combination or panel or group of drugs as identified and/or selected according to a method according to the present invention, wherein preferably said computer program is implemented on the testing system according to the present invention.

In a seventh aspect, the present invention solves the above object by providing the use of the testing system according to the present invention or computer program according to the present invention for generating at least one patient-specific drug or combination or panel or group of drugs.

In an eighth aspect, the present invention solves the above object by providing at least one patient-specific drug or combination or panel or group of drugs as identified and/or selected according to a method according to the present invention.

As mentioned above, the present invention provides an improved method for determining the effectivity of at least one, in particular personalized, drug or drug combination or panel of drugs for the treatment of a patient. In brief, the methods of the present invention advantageously take into account the pharmacokinetic parameter of a drug or group of drugs or panel of drugs to be tested, i.e. the differences in the t½, Cₘₐₓ or Tₘₐₓ or the AUC of a drug or group of drugs or panel of drugs to be tested are considered when screening for efficient drug candidates and panels for a patient. This is of particular importance in case of combinations of drugs that are then jointly tested with respect to their behavior and effect in a microtissue culture, because it reflects their behavior(s) in vivo to a much better extent than common methods. This was found to be of particular relevance and importance when comparing results between assay results obtained in mice versus human.

The inventive method first comprises the step of providing at least one culture of a 3D microtissue, such as a microtumor, based on dissociated cells of a tissue sample derived from a patient. While the main focus of the method is on a patient-specific analysis, also a pre-selected group of patients may be the donors for the cells forming the source for the microtissue(s), such as patients identified or diagnosed as having the same type of neoplastic disease.

Then, a drug or a combination of drugs or panel of drugs to be tested is provided. For this drug or a combination of drugs or panel of drugs, the t½ time period(s) is/are identified and/or determined. Respective methods are known to the person of skill in the art, and may be found in the literature. Preferred are tests in a human model. Thus, in accordance with the existing guidelines, the rate (Cₘₐₓ, tₘₐₓ) and extent (AUC) of absorption of drug from a test formulation (vs. reference formulation) is evaluated using a single dose study in healthy volunteer subjects, followed by measuring the blood/plasma concentration of the parent drug and any major active metabolites (if present) for a time period of ≥3 t½.

tₘₐₓ is usually related to the time period/length of t½, as drugs with short half-lives tend to peak and are eliminated quickly, often requiring more frequent dosing or redosing to maintain a drug within its clinically effective therapeutic range (see also below).

For a determination of the parameters, blood samples may be taken at 0, 1, 2, 3, 4, 5, 6, 7, and 8 hours, optionally followed by 10 and 12, 18, 24, 30, 36, and 42 hours after administration. As a general rule, the more frequently samples around the expected time of the maximum concentration are taken, the more accurate the value of t½ (and tₘₐₓ and Cₘₐₓ) are. The above determinations can be made in several individuals, and median values can be determined as parameter e.g. for grouping, and testing.

It is preferred that the values as used are or were determined under assay conditions that are identical or substantially identical, in order to allow maximal comparability, and therefore precision of the results. Nevertheless, in particular in more complex panels, it is envisioned to be sufficient to use values obtained from different assays without that the results of the method become insufficiently accurate or useful.

The inventive method then comprises an optional pre-selection or grouping of the combination or panel of drugs according to the t½ time periods as identified as above. This may be done in order to reduce the complexity of the assay in case of very large panels of drugs through combining drugs showing an identical or substantially or sufficiently similar t½ time periods, for example for a pre-screening of a panel.

The at least one 3D microtissue culture is then suitably contacted with the drug or combination or panel or group of drugs to be tested as identified to have the longest t½ time period(s) (T_{L1}). The end of this time period(s) define(s) the time point for a removal R of the drug or panel or group of drugs to be tested from the culture. In effect, R defines also the end of a round of all drugs or combination or panel or group of drugs to be tested. In case of several rounds or cycles of tests over a longer period of incubation (see Figures and examples as herein), R may be defined as R1, R2, . . . . Rx, wherein X defines the number of rounds or cycles for the particular set of analyses. In the context of the present invention, contacting may include introducing a suitable amount or concentration of the drug or combination or panel or group of drugs to be tested into the medium for the culture, either with the medium itself or introduced into the culture as a separate drug solution.

The at least one 3D microtissue culture, preferably an array of cultures, is then suitably incubated with the drug or combination or panel or group of drugs to be tested. Usually, the culture conditions are maintained while the drugs are in contact with the microtissuc.

The inventive method then comprises an optional step of determining an effect of said drugs and/or combinations thereof on at least one physiological parameter of the at least one 3D microtissue culture. The effect may be detected either shortly after the addition of the drugs and/or combinations thereof (e.g. within one hour after addition), or shortly before the addition of additional drugs and/or combinations thereof. This step allows to determine the individual effect of the first drug or combination of drugs as tested, before combining it/them with the next drug(s) to be tested.

In the context of the present invention, the term “at least one physiological parameter of the at least one 3D microtissue culture” shall mean any parameter of the cells of the culture and/or the microtissue that is selected and suitable to detect an effect of the drugs as tested, either individually or in combination, on said cells or tissue. Preferably, the parameter is selected from a size determination of said 3D microtissue, quantification of internal reporter gene expression in said 3D microtissue or the cells thereof (see, for example, Messner C J, Babrak L, Titolo G, Caj M, Miho E, Suter-Dick L. Single Cell Gene Expression Analysis in a 3D Microtissue Liver Model Reveals Cell Type-Specific Responses to Pro-Fibrotic TGF-β1 Stimulation. Int J Mol Sci. 2021 Apr. 22;22(9): 4372. doi: 10.3390/ijms22094372. PMID: 33922101; PMCID: PMC8122664.), determination of the intracellular ATP content in said 3D microtissue or the cells thereof, and determination of pre-selected biomarkers in said 3D microtissue and/or the cells thereof (e.g. apoptosis), wherein preferably said size determination of said 3D microtissue comprises at least one parameter selected from diameter, perimeter, volume, and area of optical cross section, and wherein preferably said size determination of said 3D microtissue comprises the use of an imaging device, and optionally further comprising the analysis of growth-kinetics. The parameters may also include tissue related parameters, like the detection of secreted/extracellular proteinaceous factors (see, for example, Otto, L., Wolint, P., Bopp, A. et al. 3D-microtissue derived secretome as a cell-free approach for enhanced mineralization of scaffolds in the chorioallantoic membrane model. Sci Rep 11, 5418 (2021). https://doi.org/10.1038/s41598-021-84123-x), or vascularization. Respective methods are known to the person of skill, and described in the literature, and herein.

The at least one 3D microtissue culture is then suitably contacted with the drug or combination or panel or group of drugs to be tested as identified to have/exhibit a shorter t½ time period(s) (T_{L2}) when compared to the drug or combination or panel or group of drugs to be tested in the step above (i.e. T_{L1}). The contacting takes place at time point R minus T_{L2}.

The at least one 3D microtissue culture, preferably an array of cultures, is then suitably incubated with the drug or combination or panel or group of drugs to be tested. Usually, the culture conditions are maintained while the drugs are in contact with the microtissue.

The inventive method then comprises a second optional step of determining an effect of said drugs and/or combinations thereof on at least one physiological parameter of the at least one 3D microtissue culture. The effect may be detected either shortly after the addition of the drugs and/or combinations thereof (e.g. within one hour after addition), or shortly before the addition of additional drugs and/or combinations thereof. This step allows to determine the joint effect(s) of the first and second drug or combination of drugs as tested, before combining it/them with the next drug(s) to be tested.

The method then optionally comprises repeating the steps of suitably contacting the at least one 3D microtissue culture, preferably an array, with at least one drug or panel combination or group of drugs to be tested identified to have a shorter t½ value or values (T_{L3}, T_{L4}, . . . , etc.) compared to the drug or panel or group of drugs to be tested in the steps beforehand at time points R minus T_{L3}, R minus T_{L4}, . . . , etc, and suitably incubating the at least one 3D microtissue culture, preferably an array, with the drug or combination or panel or group of drugs to be tested. Usually, the culture conditions are maintained while the drugs are in contact with the microtissue.

At timepoint R (or R1, R2, R3, . . . , etc.) the drug or panel or combination or group of drugs to be tested is/are removed from the at least one 3D microtissue culture, preferably the array. This is usually done by replacing the medium with fresh medium/replacement medium or suitable buffers not containing drugs to be tested. Nevertheless, depending on the analysis and results as desired, it is possible to remove the drugs by replacing the medium with fresh medium/replacement medium or suitable buffers containing a new drug or panel or combination or group of drugs to be tested.

The inventive method then comprises the step of determining an effect of said drugs and/or combinations thereof on at least one physiological parameter of the at least one 3D microtissue culture. The effect may be detected shortly after the removal of the drugs and/or combinations thereof (e.g. within one hour after removal), or shortly before the beginning of a new cycle as mentioned above, i.e. the addition of new drugs and/or combinations thereof to be tested within R2, R3, . . . , etc. This step allows to determine the joint effect(s) of the drug or combination of drugs as tested. The effect(s) of the drugs then show the effectivity of at least one, in particular personalized, drug or drug combination for the treatment of a patient, or further allow to directly conclude on the effectivity of the at least one, in particular personalized, drug or drug combination for the treatment of a patient.

Preferred is then the method according to the present invention, further comprising the step of identifying at least one patient-specific (effective) drug or combination or panel or group of drugs based on the effect(s) as determined. As mentioned above, it was found that the present method identifies patient-specific (effective) drugs or combination or panel or group of drugs with a much higher in vivo relevance than common models. Further preferred is then the method according to the present invention, further comprising the step of selecting said patient-specific drug or combination or panel or group of drugs as identified. This provides highly efficient and patient-specific (and disease-specific) combinations or panels or group of drugs that represent advantageous tools for treating and preventing the diseases as described herein.

Preferred is the method according to the present invention, wherein R (or R1, R2, . . . , etc.) furthermore includes the time to tₘₐₓ for the drug or combination or panel or group of drugs as tested, i.e. R=tₘₐₓ+t½. The inclusion has the additional advantage to even further reflect the actual in vivo situation. For a comparability of the values as determined and used, it is preferred to either include the time to tₘₐₓ for all drugs or combinations or panels or group of drugs as tested, or to not include the value for all drugs/values as used. This is particularly the case, if the tₘₐₓ for a drug as tested is not (readily) available.

Further preferred is a method according to the present invention, wherein the at least one culture of a 3D microtissue is contacted with the drug or combination or panel or group of drugs at the Cₘₐₓ concentration. This ensures that the results are obtained more or less independently of the (bio-) availability of the drugs at the site of action. Again, for a comparability of the values as determined and used, it is preferred to either include the Cₘₐₓ concentration for all drugs or combinations or panels or group of drugs as tested, or to not include the value for all drugs/values as used. This is particularly the case, if the Cₘₐₓ concentration for a drug as tested is not (readily) available.

Further preferred is a method according to the present invention, wherein identifying the t½ value(s) for the drug or combination or panel of drugs to be tested comprises testing the drug in a subject or a group of subjects, or comprises identifying said t½ from the literature or a database. As mentioned above, the t½ time period(s) may be or is/are identified and/or determined. Respective methods are known to the person of skill in the art, and may be found in the literature. Preferred are tests in a human model. Thus, in accordance with the existing guidelines, the rate (Cₘₐₓ, tₘₐₓ) and extent (AUC) of absorption of drug from a test formulation (vs. reference formulation) is evaluated using a single dose study in healthy volunteer subjects, followed by measuring the blood/plasma concentration of the parent drug and any major active metabolites (if present) for a time period of ≥3 t½. tₘₐₓ is usually related to the time period/length of tin, as drugs with short half-lives tend to peak and are eliminated quickly, often requiring more frequent dosing or redosing to maintain a drug within its clinically effective therapeutic range (see also below). For a determination of the parameters, blood samples may be taken at 0, 1, 2, 3, 4, 5, 6, 7, and 8 hours, optionally followed by 10 and 12, 18, 24, 30, 36, and 42 hours after administration. As a general rule, the more frequently samples around the expected time of the maximum concentration are taken, the more accurate the value of t½ (and tₘₐₓ and Cₘₐₓ) are. The above determinations can be made in several individuals, and median values can be determined as parameter e.g. for grouping, and testing.

According to another aspect of the present invention, the panel or combination of drugs is composed, selected and/or established at least in part, and preferably completely or substantially completely based on the t½ values as identified in step c) of the method according to the present invention as described herein. This aspect relates to the pre-grouping of compounds in cases where several or many drugs shall be tested. The pre-grouping reduces the complexity of the assay, and may also be used as a pool in a first “run” of the method in order to identify drugs that may be promising candidates for further and more detailed analyses.

In the method according to the present invention, the drugs to be tested can also be provided to the test as a pre-manufactured drug matrix. Such a matrix can be based on the indication as analysed, the cells and tissues as used, and/or the class of drugs, i.e. the chemical class or other class of molecules (e.g. antibodies, proteins, chemical “small” molecules, and the like), or even distinguish between registered and non-registered or natural compound (e.g. in a library).

Thus, further preferred is a method according to the present invention, wherein said contacting particularly in step e) or g) comprises an exposure to one drug, or simultaneously to at least two drugs and/or combinations of drugs.

In general, the method according to the present invention can be performed over any suitable period of time, which is for example controlled or determined by the cells and tissues and/or the physical parameters of the drugs to be tested, in particular the t½. The present method provides preferably for longer incubation times, and preferred is a method, wherein the incubation time is between 5 to 50, preferably between 6 to 35, and most preferred between 7 to 28 days. The incubation as mentioned can be either determine a single “cycle” of drugs to be tested, and optionally repeated, or can indicate the combined overall incubation time.

The drugs to be tested can be selected from any suitable drugs, depending on the underlying tape of disease and/or the cell and tissue type to as used. Preferred is the method according to the present invention, wherein the drugs to be tested are selected from anti-cancer drugs, such as, for example, alkylating agents, antimetabolites, natural products, hormones, tyrosine inhibitors, chemotherapeutic compounds, anti-cancer antbodies, in particular Gemcitabine, Abraxame, Trametinib, Olaparib, Oxaliplatin, Erlotinib, Erlotinib, 5-FU, Docetaxel, and Pemetrexed. The drugs to be tested can further be selected from the group consisting of cytotoxic, cytostatic and/or chemotherapeutic agents, targeted drugs, immunotherapeutic agents and/or combinations thereof. Examples for cytotoxic, cytostatic and/or chemotherapeutic agents are Anastrozole, Azathioprine, Bcg, Bicalutamide, Chloramphenicol, Ciclosporin, Cidofovir, Coal tar containing products, Colchicine, Danazol, Diethylstilbestrol, Dinoprostone, Dithranol containing products, Dutasteride, Estradiol, Exemestane, Finasteride, Flutamide, Ganciclovir, Gonadotrophin, chorionic, Goserelin, Interferon containing products (including peg-interferon), Leflunomide, Letrozole, Leuprorelin acetate, Medroxyprogesterone, Megestrol, Menotropins, Mifepristone, Mycophenolate mofetil, Nafarelin, Oestrogen containing products, Oxytocin (including syntocinon and syntometrine), Podophyllyn, Progesterone containing products, Raloxifene, Ribavarin, Sirolimus, Streptozocin, Tacrolimus, Tamoxifen, Testosterone, Thalidomide, Toremifene, Trifluridine, Triptorelin, Valganciclovir, and Zidovudine. Targeted drugs are medications that increase in concentration in some parts of the body relative to others, such as antibodies. Examples are in brain cancer: bevacizumab, everolimus; breast cancer: bevacizumab, everolimus, lapatinib, pertuzumab, trastuzumab and its antibody drug conjugates; in colorectal cancer: aflibercept, bevacizumab, cetuximab, panitumumab, regorafenib, and dermatofibrosarcoma protuberans: imatinib. Immunotherapeutic agents are used in immunotherapy that is a form of cancer treatment that uses the power of the body's immune system to prevent, control, and eliminate cancer. Examples are monoclonal antibodies to treat cancer, CAR T-cell therapy, immune checkpoint inhibitors to treat cancer, cancer vaccines, immunomodulating drugs (IMiDs), and cytokines.

In the method according to the present invention, the patient preferably is a mammalian patient, in particular a human, and wherein preferably said patient suffers from, or is being diagnosed for, a neoplastic disease or tumor. Consequently, the microtissue and/or cells as used in the assays of the invention are mammalian cells, in particular human cells. The cells as used in the method of the present invention are thus usually autologous to a particular patient that is to be treated. Nevertheless, cells may also be used that represent a group of patient (e.g. suffering from a particular neoplastic disease or group of diseases, like lung or colon cancer), and it may also be useful to include cells based on cancer cell lines as available. Other cells may be included, too. The addition or removal of cells helps controlling the microtissue environment. Particularly preferred is the addition of immune cells that are known to interact with tumors and cells in the tumor and/or are known to invade tumors in vivo, e.g. so-called tumor-infiltrating immune cells. Examples are PBMCs, lymphocytes, recombinant T cells, and the like. These settings and strategies are of particular advantage for tests and assay in the context of cancer-immunotherapy, and can furthermore involve the addition of other factors, like cytokines etc. to the microtissue environment. Both cells and factors can be added also after the formation of the mnicrotissue(s). Thus, in another embodiment of the method according to the present invention, step a) comprises adding or removing stroma cells, stromal fibroblasts, endothelial cells and immune cells to said dissociated cells, and/or wherein in step a) for each 3D microtissue a predetermined number of cells is provided, such as, for example, between 500 and 10000 viable cells, and/or wherein in step a) said 3D microtissues are generated in at least one system selected from a hanging drop system, and a multiwell system, preferably comprising Ultra Low Adherence (ULA) wells.

In the method according to the present invention, the patient-derived tissue sample is selected from a sub-sample derived from a primary tissue sample, a primary tumor sample, and a metastasis sample, and wherein preferably said tissue sample has been obtained by a method comprising core biopsy, tumor resection, liquid biopsy and/or needle aspiration, as well as other biopsies, surgery, and lavage. The tissue sample can be obtained from a tumor selected from the group of confirmed or suspected neoplastic or cancerous tissues comprising neoplastic liver tissue, neoplastic kidney tissue, neoplastic skin tissue, neoplastic prostate tissue, neoplastic breast tissue, neoplastic ovary tissue, neoplastic brain tissue, etc., in particular from neoplastic liver tissue, particularly from a primary or a metastatic liver tumor.

In an embodiment of the method according to the present invention, step a) comprises dissociating said patient-derived tissue sample comprising i) optionally, physically dissecting said tissue sample into smaller pieces comprising cells, ii) treating said tissue sample with a solution comprising at least one enzyme capable of dissociating cells in said tissue sample, preferably at least one enzyme selected from a protease, a collagenase, trypsin, elastase, hyaluronidase, papain, chymotrypsin, deoxyribonuclease I, and neutral protease (dispase), producing a supernatant comprising dissociated cells, and iii) removing said supernatant comprises said dissociated cells and suitably collecting said cells. Preferably, steps (ii) and (iii) are repeated at least once, wherein preferably before step (ii) said tissue sample is sonicated with ultrasound, wherein the energy of said ultrasound is set at a suitable level to not destroy a substantial amount of said cells.

As mentioned above, any parameter of the cells of the culture and/or the microtissue that is selected and suitable to detect an effect of the drugs as tested, either individually or in combination, on said cells or tissue. Preferred is the method according to the present invention, wherein said determining of said physiological parameter in step f) and/or k) is selected from size determination of said 3D microtissue, quantification of internal reporter gene expression in said 3D microtissue, determination of the intracellular ATP content in said 3D microtissue, and determination of pre-selected biomarkers in said 3D microtissue, wherein preferably said size determination of said 3D microtissue comprises at least one parameter selected from diameter, perimeter, volume, and area of optical cross section, and wherein preferably said size determination of said 3D microtissue comprises the use of an imaging device, and optionally further comprising the analysis of growth-kinetics.

In an embodiment of the method according to the present invention, only a fraction/aliquot of the cells is analyzed, and wherein said method optionally comprises providing a primary tissue sample, obtaining a subsample in addition to the patient-derived sample and subjecting said subsample to at least one of molecular profiling, histological analysis, and histochemical analysis. This provide additional information for the overall analysis. Preferred is a method according to the present invention, wherein said step of identifying and/or selecting includes the use of additional clinical parameters, such as patient specific data and/or treatment history.

In yet another aspect thereof, the present invention provides a method for identifying adverse effects associated with a treatment with a patient-specific drug or combination or panel or group of drugs in a patient, comprising performing the method according to the present invention as above, and further comprising the step of testing and analyzing said patient-specific drug or combination or panel or group of drugs for adverse effects in said patient. An adverse effect in the form of an adverse drug reaction (ADR) is an appreciably harmful or unpleasant reaction, resulting from an intervention related to the use of a medicinal product (drug), which predicts hazard from future administration and warrants prevention or specific treatment, or alteration of the dosage regimen, or withdrawal of the product (see, for example, Edwards and Aronson, The Lancet, 356, 1255-1259).

In yet another aspect thereof, the present invention provides a method for stratifying a patient with respect to a treatment with a patient-specific drug or combination or panel or group of drugs, comprising performing the method according to the present invention, comprising identifying at least one patient-specific drug or combination or panel or group of drugs based on the effect(s) as determined, and stratifying of said patient based on said patient-specific drug or combination or panel or group of drugs as identified, preferably into different patient and/or treatment groups.

In yet another aspect thereof, the present invention provides a method for monitoring the treatment with a patient-specific drug or combination or panel or group of drugs, comprising performing the method according to the present invention, treating said patient with said at least one patient-specific drug or combination or panel or group of drugs as identified, and repeating the method according to the present invention on a sample of said patient as treated, and optionally adjusting the treatment based on the result(s) of said repeating.

Preferred is a method according to the present invention, wherein said method is at least in part, and preferably fully, automated, comprising the use of at least one robot and/or computing apparatus. Preferably, the workflow of the inventive method is designed to be fully compatible with automated liquid handling processes.

In yet another aspect thereof, the present invention provides a testing system comprising means for performing the method according to the present invention, comprising a) a unit for culturing an array of 3D microtissues, such as microtumors, based on dissociated cells of a tissue sample derived from the patient as provided, b) a drug testing unit for contacting said array of said 3D microtissues with at least two drugs or combination or panel or group of drugs to be tested in accordance with their respective t½ time period(s) (T_{L1}, and/or T_{L2}, TL₃, . . . ), c) a first analysis unit for determining an effect of said drugs or combination or panel or group of drugs thereof on said array of said 3D microtissues, and d) a second drug testing unit for removing the drug or panel or combination or group of drugs to be tested from the culture at timepoint R.

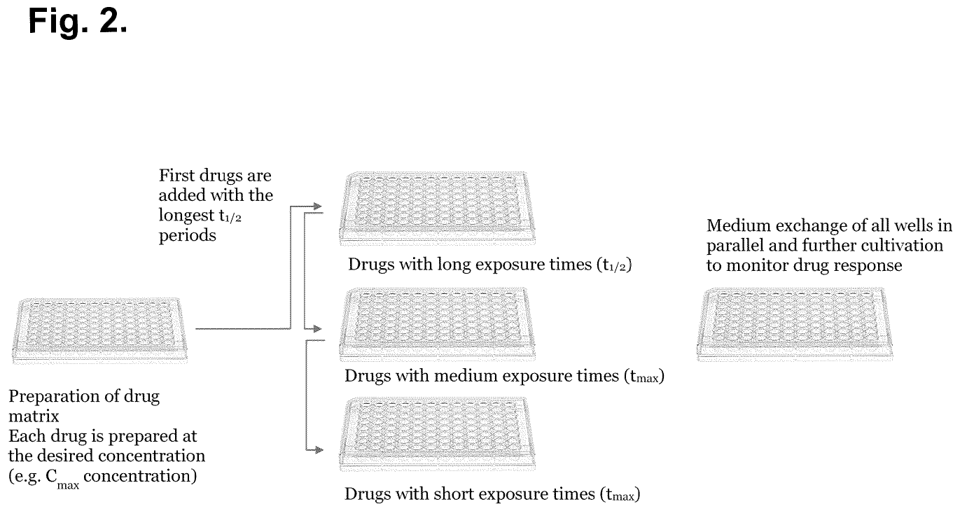

The testing system thus comprises physical means for suitably performing the steps of the method. Advantageously, the testing system is composed of units that are designed to perform certain steps of the method, as explained below. Preferably, the system can be sterilized as a whole or in parts (e.g. units) thereof, and/or wherein said system comprises means for establishing and/or maintaining sterile conditions. Examples of the design and layout of testing systems and units thereof are known to the person of skill, and are, for example, described in FIGS. 2 to 6 of WO2021/110799.

Preferred embodiments of the testing system according to the present invention, for example, further comprise at least one incubation timer and/or at least one drug reservoir, and/or tissue sample dissociation unit.

Preferably, said tissue sample dissociation unit comprises at least one of i) a pipetting unit, ii) an enzyme reservoir, iii) a reservoir for cell culture media, iv) a reservoir for washing solutions, v) optionally, an ultrasonic device, and vi) a centrifuge unit, and/or wherein said unit for producing an array of 3D microtissues, such as microtumors, based on said dissociated cells comprises at least one of i) a pipetting unit, ii) a cell counting unit, and, iii) a handler for microtiter plates, and/or wherein said drug testing unit comprises at least one of i) a handler for microtiter plates, ii) a pipetting unit, iii) a reservoir for cell culture media, iv) an array of reservoirs comprising at least two different drugs or combinations thereof, and iv) an incubator unit, and/or wherein said first and/or second analysis unit comprises i) a handler for microtiter plates, and/or ii) an imaging system comprising a microscope and a camera, and optionally an HR scanner.

In preferred embodiments of the testing system according to the present invention, the tissue sample dissociation unit and said unit for the production of an array of 3D microtissues share the same pipetting unit and/or wherein said drug testing unit and said first analysis unit share the same handler for microtiter plates.

In preferred embodiments of the testing system according to the present invention, the tissue sample dissociation unit and said unit for producing an array of 3D microtissues are positioned in the same housing, and/or wherein said drug testing unit and said first analysis unit are positioned in the same housing, and preferably wherein said two housings are connected to form a discrete system and/or wherein said system is, at least in part, arranged vertically.

In another aspect of the testing system according to the present invention, before step a), the tissue sample is sonicated with ultrasound, for example pulsed ultrasound. This step is performed to induce hemolysis, wherein the energy of said ultrasound is set at a suitable level without destroying and/or damaging a substantial amount of said cells to be analyzed, for example at about 1 MHz, 0.5-5, and preferably 2 Wcm⁻², i.e. milder than pro preparing DNA or RNA from a cellular sample. The hemolysis step using sonification optimally lasts for about five minutes, and targets cells that are not embedded in the tissue environment.

In another aspect of the testing system according to the present invention, the system comprises at least one loading port comprising a loading system with a lock system (L) for a sterile loading of materials or consumables as used in the system(s) and/or unloading waste and/or products as produced in the system(s). Said lock system may preferably further comprises means for sterilizing the materials, such as, for example, by UV, and/or wherein said lock system further comprises means for thawing or cooling/freezing the materials to be loaded or unloaded. More preferably, the lock system is adapted to specifically fit to a transport box or container, wherein preferably said transport box or container comprises at least one port to be opened and closed inside the system. The transport box or container may comprise at least two different separate temperature zones, such as zones that are kept at −20° C., 4° C., or ambient temperature, and wherein optionally said transport box or container is insulated.

In another aspect of the testing system according to the present invention, the testing system further comprises at least one means for determining physiological parameter selected from size determination of said 3D microtissue, quantification of internal reporter gene expression in said 3D microtissue, determination of the intracellular ATP content in said 3D microtissue, and determination of pre-selected biomarkers in said 3D microtissue, wherein preferably said means for size determination of said 3D microtissue comprise means for at least one parameter selected from diameter, perimeter, volume, and area of optical cross section, preferably imaging devices, and means for the analysis of growth-kinetics. Further to what is mentioned above, the size determination of said 3D microtissue may comprise the use of an optical method, such as using an imaging device. Particularly useful is high-resolution scanning electron microscopy (HR-SEM).

For method the analysis of growth-kinetics of said cells and microtissue, growth kinetics is an autocatalytic reaction which implies that the rate of growth is directly proportional to the concentration of cells. The cell concentration can be measured by direct and indirect methods that are known to person of skill, and described in the literature (cell count or optical density/turbimetry).

In another aspect of the testing system according to the present invention, the testing system further comprises means for identifying at least one patient-specific drug or combination or panel or group of drugs based on the effect(s) as determined, and, optionally, further comprising means for selecting said patient-specific drug or combination or panel or group of drugs as identified.

In one preferred embodiment, the testing system according to the present invention comprises at least one database for collecting and/or storing data selected from the group consisting of t½ time periods, data with respect to the pre-selection and/or grouping of the combination or panel of drugs, physiological parameters, data with respect to the effect(s) of said drugs or combination or panel or group of drugs thereof, data with respect to adverse effects, data with respect to additional clinical parameters, such as patient specific data and/or treatment history, data with respect to the stratification and/or monitoring, and data for automatization of the system, for example for controlling at least one robot.

Additional data in the database may be selected from i) data about the molecular profile of said tissue sample, ii) results of a histological analysis of said tissue sample, iii) results of a histochemical analysis of said tissue sample, iv) patient anamnesis, e.g. selected from the group comprising checking/determining vital functions, patient history, smoking habits, sports activities, hormone levels, previous or current medications, etc., v) hereditary information regarding the patient, and vi) genomic information for the patient.

Another aspect of the present invention then relates to a computer program comprising means for controlling the testing system according to the present invention, in particular a computer program comprising data for at least one patient-specific drug or combination or panel or group of drugs as identified and/or selected according to a method according to the present invention, wherein preferably said computer program is implemented on the testing system according to the present invention.

Another aspect of the present invention then relates to the use of the testing system according to the present invention or computer program according to the present invention for generating at least one patient-specific drug or combination or panel or group of drugs.

Yet another aspect of the present invention then relates to at least one patient-specific drug or combination or panel or group of drugs as identified and/or selected according to a method according to the present invention. This combination may be a physical set of selected drugs in a kit or combination, preferably suitable to perform a method as disclosed herein or for use to treat a patient, in particular as patient-specific treatment, or a set of data, e.g. encoding a panel of patient-specific and efficient drugs.

Another aspect then relates to method of treating a patient that suffers from, or is being diagnosed for, a neoplastic disease or tumor, comprising performing the method according to the present invention, and the step of suitably treating said patient with a patient-specific, in particular personalized, drug or drug combination as identified.

Preferred is a method according to the present invention, wherein said method provides a recommendation with regard to a suitable patient-or patient-group-specific drug or drug combination in order to more effectively treat the patient suffering from, or being diagnosed for, a given neoplastic disease or tumor.

The method can furthermore take into account a method according to the present invention for stratifying a patient with respect to a treatment with a patient-specific drug or drug combination, comprising performing the method according to the present invention as above, and further comprising a stratification of said patient based on said patient-specific drug or drug combination as identified, preferably into different patient and/or treatment groups, and/or a method for identifying adverse effects associated with a treatment with a patient-specific drug or drug combination in a patient, comprising performing the method according to the present invention as above, and further comprising the step of testing and analyzing said patient-specific drug or drug combination for adverse effects in said patient.

The present invention analyzes tissue biopsy samples of a patient or preselected group of patients that suffer from, or are diagnosed for, a neoplastic disease or tumor. These samples are used to generate 3D microtissues, in particular so-called “microtumors” that are then used in testing for effective and optimally patient-specific effective therapeutic drugs of combinations of drugs for a treatment and/or the prevention of said neoplastic disease or tumor. The present methods and systems allow for a fast analysis and search for drugs, and in particular screens for drugs and drug combinations that are effective for the actual patient and/or specific patient-group.

The methods and system further have the advantage to closely mimic the actual situation in vivo, and utilizing 3D tissue models allows for extended assay times (7-28 days) to assess safety and efficacy as well as to incorporate differential pharmacokinetic properties of drugs. Thus, a process has been developed to minimize complexity and increase assay robustness of dealing with individual drug exposure times for testing multiple drugs in parallel. Further, the tests allow to add cells to create a microtumor-environment that cannot be achieved by regular screening methods using single cell-based screens. Further, the method can be “accompanied” by the analysis of a so-called subsample in addition to the patient-derived sample, and subjecting said subsample to at least one of molecular profiling, histological analysis, and histochemical analysis, as also explained further herein.

It was found by the present inventors that the correct dosing of drugs has an enormous impact on the comparability of drug testing assays. Since the process is mostly designed in the direction mouse to human, mouse blood concentrations often are found to be not comparable to the situation in human.

In the context of the present invention, a 3D microtissue shall designate an in vitro generated cell aggregate comprising cells as desired. Consequently, a microtumor shall mean a 3D microtissue generated from, at least in part, selected cancer cells derived from a cell line or a neoplastic sample, such as a tumor (see, for example, Rimann et al., An in vitro osteosarcoma 3D microtissue model for drug development, Volume 189, 10 Nov. 2014, Pages 129-135). These two terms herein are used interchangeably.

In the context of the present invention, an “array” is a set of separate 3D microtissues that tested/analyzed, such as, for example, 3D microtumors in a multi-well plate. An array are at least 2 or more, or 3, 4, 5, 6, 7, 8, 9, 10 or more, 12, 48, 96, 128, 384 or more, or even 200, 300, 400, or more 3D microtumors/microtissues.

Preferred is a method according to the present invention, wherein for each 3D microtissue a predetermined number of cells is provided, for example about 500, about 1000, about 2000, about 5000 or about 10000 cells, in particular viable cells. This further can comprise suitably counting the cells prior to the generation of said microtissues, preferably using a suitable automated cell counting unit (e.g. from Logos Biosystems, South Korea).

Preferred is a method according to the present invention, wherein the production of the 3D microtissues does not require the use of a solubilized basement membrane preparation, like Matrigel®. This has the advantage that the complete method can be performed at temperatures above 4° C., such as room temperature. Preferred is a method according to the present invention, wherein the generation of said 3D microtissues comprises self-assembly of said cells comprised in said dissociated cells.

Further preferred is a method according to the present invention, wherein the generation of said 3D microtissues comprises a maturation time of about 6 hours to 7 days, preferably about 1 to 6 days, more preferably about 2 to 5 days. Further preferred is a method according to the present invention, wherein the 3D microtissues as generated have a size of about 350 μm +/−100 μm. Desired and preferred is a size that is suitable for a proper analysis, in particular for the optical analysis methods as disclosed herein.

A particular important aspect of the system and method according to the present invention is automation, which provides fast and reproducible results. Preferred is therefore a system according to the present invention, wherein at least one unit thereof, and preferably substantially all units thereof, function in an automated manner.

In another aspect of the system according to the present invention, the system can be sterilized as a whole or in parts thereof and/or comprise means for establishing and/or maintaining sterile conditions, such as sterilized by UV irradiation, ozone treatment, radiation, and/or includes sections with laminar flow, etc.

The terms “of the (present) invention”, “in accordance with the invention”, “according to the invention” and the like, as used herein are intended to refer to all aspects and embodiments of the invention described and/or claimed herein.

The terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

As used herein, the term “sample” refers to a clinical sample obtainable from a patient, such as a patient suspected to have a neoplastic disease, e.g., a tumor/cancer, or a patient with confirmed neoplastic disease, e.g., a tumor/cancer. The sample may be obtained by any known type of biopsy, e.g., needle biopsy, liquid biopsy, core biopsy, tumor resection, needle aspiration, surgical removal of solid neoplastic tissue, as known in the art.

As used herein, the term “treatment” refers to any type of therapeutic intervention, including curing a disease, alleviation of a disease, improvement of the disease, slowing down the progression of a disease, and the like, as well as the alleviation or improvement or suppression of disease symptoms, such as pain, and the like, in particular are those associated with neoplastic diseases/cancer.

As used herein, the term “prevention” refers to any type of intervention that is suitable of preventing the development of a disease, particularly a neoplastic disease, or that is capable of slowing down, inhibiting the worsening of a disease or disease symptoms, particularly neoplastic diseases/cancer.

As used herein, the term “neoplastic” refers to any growth of tissue, which has lost growth control, including solid or liquid tumors, warts, metastatic growth, etc.

As used herein, the term “tumor” refers to any benign or malignant tissue in any given organ system or tissue, for example, liver, kidney, brain, breast, prostate, skin, etc.

As used herein, the term “microplate” refers to any arrangement of multiple cavities that can be used as reaction vessels or culture vessels, for example 24 well plates, 48 well plates, 96 well plates, 384 well plates, etc. as known in the art. It is also contemplated to use and arrangement of individual vessels, which do not necessarily have to be arranged as a plate, but may also be arranged informal strips of individual vessels, etc.

As used herein, the term “sonification” relates to the treatment of a tissue or cells with ultrasound to thereby destroy the integrity of certain target structures such as red blood cells in a process called hemolysis. This permits the removal from the tissue sample of hemoglobin, which can disturb the analysis of the obtained cells, particularly the optical analysis of cells due to interference of the hemoglobin with the detection means. Hemolysis can also be achieved by incubation of tissue in solutions of very high or very low osmolality inducing the burst or shrinkage of the red blood cells, by treatment with certain hemolytic enzymes, etc.

As used herein, the term “patient” refers to a human or non-human patient. The non-human patient may preferably be a mammal, for example, a horse, a dog, a cat, etc., that have been diagnosed with or are suspected to have a neoplastic disorder/cancer.

As used herein, the term “drug” refers to any active agent, small molecule or biotechnologically-produced molecule or combination of molecules, nucleic acid construct(s), e.g. a vector for gene therapy, the effect of which shall be tested in vitro using the herein described methods and means.

As used herein, the term “stratifying” refers to the process in which a given individual/patient is classified into a group that shall be treated in a particular way.

As used herein, the term “adverse effect” in the present context refers to any unwanted side-effect associated with the exposure of a microtissue as defined herein to a given compound or drug or combination thereof and may comprise, inter alia, promotion of cell growth, resistance development to a given drug, loss of expression of MHC-expression, surface-expression and/or secretion of factors down-regulating the immune system, etc., all of which indicate that the sample-derived cells appear to be less affected by/become resistant to the exposure of a given compound.

The present invention in particular relates to the following preferred Items.

Item 1. A method for determining the effectivity of at least one, in particular personalized, drug or drug combination for the treatment of a patient, said method comprising a) providing at least one culture of a 3D microtissue, such as a microtumor, based on dissociated cells of a tissue sample derived from a patient, b) providing a drug or a combination or panel of drugs to be tested, c) identifying the t½ time period(s) for said drug or combination or panel of drugs of step b), e) contacting the at least one 3D microtissue culture with the drug or combination or panel or group of drugs to be tested identified to have the longest t½ time period(s) (T_{L1}), wherein the end of this time period(s) define(s) the time point for a removal R of the drug or panel or group of drugs to be tested from the culture, f) incubating said at least one 3D microtissue culture, preferably an array of cultures, with the drug or combination or panel or group of drugs to be tested, and, optionally, determining an effect of said drugs and/or combinations thereof on at least one physiological parameter of the at least one 3D microtissue culture, g) subsequent to e), contacting the at least one 3D microtissue culture with the drug or combination or panel or group of drugs to be tested identified to have a shorter t½ time period(s) (T_{L2}) compared to the drug or combination or panel or group of drugs to be tested in step e) at time point R minus T_{L2}, h) incubating the at least one 3D microtissue culture with the drugs or combination or panel or group of drugs to be tested, j) removing the drug or panel or combination or group of drugs to be tested from the culture at timepoint R, and k) determining an effect of said drugs and/or combinations thereof on at least one physiological parameter of the at least one drug or panel or combination or group of drugs on the at least one 3D microtissue culture.

Preferred is Item 1 as above, optionally comprising at least one of d) pre-selecting or grouping the combination or panel of drugs according to the t½ time periods as identified in step c); i) repeating steps g) and h) with at least one drug or panel combination or group of drugs to be tested identified to have a shorter t½ value or values (T_{L3}, T_{L4}, . . . ) compared to the drug or panel or group of drugs to be tested in the steps beforehand at time points R minus T_{L3}, R minus T_{L4}, . . . , and/or 1) identifying at least one patient-specific drug or combination or panel or group of drugs based on the effect(s) as determined, and, optionally, further comprising the step of selecting said patient-specific drug or combination or panel or group of drugs as identified.

Item 2. The method according to Item 1, wherein R furthermore includes the time to tₘₐₓ for the drug or combination or panel or group of drugs, i.e. R=tₘₐₓ +t½.

Item 3. The method according to Item 1 or 2, wherein the at least one culture of a 3D microtissue is contacted with the drug or combination or panel or group of drugs at the Cₘₐₓ concentration.

Item 4. The method according to any one of Items 1 to 3, wherein identifying the T½ value(s) for the drug or combination or panel of drugs to be tested comprises testing the drug in a subject or a group of subjects, or comprises identifying said T½ from the literature or a database.

Item 5. The method according to any one of Items 1 to 4, wherein the panel or combination of drugs is established at least in part based on the T½ values as identified in step c)

Item 6. The method according to any one of Items 1 to 5, wherein said contacting in step e) or g) comprises an exposure to one drug, or simultaneously to at least two drugs and/or combinations.

Item 7. The method according to any one of Items 1 to 6, wherein the incubation time is between 7 to 28 days.

Item 8. The method according to any one of Items 1 to 7, wherein the drugs to be tested are selected from anti-cancer drugs, such as, for example, alkylating agents, antimetabolites, natural products, hormones, tyrosine inhibitors, chemotherapeutic compounds, anti-cancer antbodies, in particular Gemcitabine, Abraxame, Trametinib, Olaparib, Oxaliplatin, Erlotinib, Erlotinib, 5-FU, Docetaxel, and Pemetrexed.

Item 9. The method according to any one of Items 1 to 8, wherein the drugs to be tested are provided to the test as a pre-manufactured drug matrix.

Item 10. The method according to any one of Items 1 to 9, wherein the patient is a mammalian patient, in particular a human, and wherein preferably said patient suffers from, or is being diagnosed for, a neoplastic disease or tumor.

Item 11. The method according to any one of Items 1 to 10, wherein said patient-derived tissue sample is selected from a sub-sample derived from a primary tissue sample, a primary tumor sample, and a metastasis sample, and wherein preferably said tissue sample has been obtained by a method comprising core biopsy, tumor resection, liquid biopsy and/or needle aspiration, and/or wherein said tissue sample and/or the dissociated cells are frozen and re-thawed prior to the generation of said 3D microtissues.

Item 12. The method according to any one of Items 1 to 11, wherein step a) comprises dissociating said patient-derived tissue sample comprising i) optionally, physically dissecting said tissue sample into smaller pieces comprising cells, ii) treating said tissue sample with a solution comprising at least one enzyme capable of dissociating cells in said tissue sample, preferably at least one enzyme selected from a protease, a collagenase, trypsin, elastase, hyaluronidase, papain, chymotrypsin, deoxyribonuclease I, and neutral protease (dispase), producing a supernatant comprising dissociated cells, and iii) removing said supernatant comprises said dissociated cells and suitably collecting said cells.

Item 13. The method according to Item 12, wherein steps (ii) and (iii) are repeated at least once, wherein preferably before step (ii) said tissue sample is sonicated with ultrasound, wherein the energy of said ultrasound is set at a suitable level to not destroy a substantial amount of said cells.

Item 14. The method according to any one of Items 1 to 13, wherein step a) comprises adding or removing stroma cells, stromal fibroblasts, endothelial cells and immune cells to said dissociated cells, and/or wherein in step a) for each 3D microtissue a predetermined number of cells is provided, such as, for example, between 500 and 10000 viable cells, and/or wherein in step a) said 3D microtissues are generated in at least one system selected from a hanging drop system, and a multiwell system, preferably comprising Ultra Low Adherence (ULA) wells.

Item 15. The method according to any one of Items 1 to 14, wherein said determining of said physiological parameter in step f) and/or k) is selected from size determination of said 3D microtissue, quantification of internal reporter gene expression in said 3D microtissue, determination of the intracellular ATP content in said 3D microtissue, and determination of pre-selected biomarkers in said 3D microtissue, wherein preferably said size determination of said 3D microtissue comprises at least one parameter selected from diameter, perimeter, volume, and area of optical cross section, and wherein preferably said size determination of said 3D microtissue comprises the use of an imaging device, and optionally further comprising the analysis of growth-kinetics.

Item 16. The method according to any one of Items 1 to 15, wherein only a fraction/aliquot of the cells is analyzed, and wherein said method optionally comprises providing a primary tissue sample, obtaining a subsample in addition to the patient-derived sample and subjecting said subsample to at least one of molecular profiling, histological analysis, and histochemical analysis.

Item 17. A method for identifying adverse effects associated with a treatment with a patient-specific drug or combination or panel or group of drugs in a patient, comprising performing the method according to any one of Items 1 to 16, further comprising the step of testing and analyzing said patient-specific drug or combination or panel or group of drugs for adverse effects in said patient.

Item 18. A method for stratifying a patient with respect to a treatment with a patient-specific drug or combination or panel or group of drugs, comprising performing the method according to any one of Items 1 to 17, comprising identifying at least one patient-specific drug or combination or panel or group of drugs based on the effect(s) as determined, and stratifying of said patient based on said patient-specific drug or combination or panel or group of drugs as identified.

Item 19. A method for monitoring the treatment with a patient-specific drug or combination or panel or group of drugs, comprising performing the method according to any one of Items 1 to 18, treating said patient with said at least one patient-specific drug or combination or panel or group of drugs as identified, and repeating the method according to any one of claims 1 to 18 on a sample of said patient as treated, and optionally adjusting the treatment based on the result(s) of said repeating.

Item 20. The method according to any one of Items 1 to 19, wherein said step of identifying and/or selecting includes the use of additional clinical parameters, such as patient specific data and/or treatment history.

Item 21. The method according to any one of Items 1 to 20, wherein said method is at least in part, and preferably fully, automated, comprising the use of at least one robot and/or computing apparatus.

Item 22. A testing system comprising means for performing the method according to any one of Items 1 to 21, comprising a) a unit for culturing an array of 3D microtissues, such as microtumors, based on dissociated cells of a tissue sample derived from the patient as provided, b) a drug testing unit for contacting said array of said 3D microtissues with at least two drugs or combination or panel or group of drugs to be tested in accordance with their respective t½ time period(s) (T_{L1}, and/or T_{L2}, TL₃, . . . ), c) a first analysis unit for determining an effect of said drugs or combination or panel or group of drugs thereof on said array of said 3D microtissues, and d) a second drug testing unit for removing the drug or panel or combination or group of drugs to be tested from the culture at timepoint R.

Item 23. The testing system according to Item 22, further comprising means for identifying at least one patient-specific drug or combination or panel or group of drugs based on the effect(s) as determined, and, optionally, further comprising means for selecting said patient-specific drug or combination or panel or group of drugs as identified.

Item 24. The testing system according to Item 22 or 23, further comprising at least one database for collecting and/or storing data selected from the group consisting of t½ time periods, data with respect to the pre-selection and/or grouping of the combination or panel of drugs, physiological parameters, data with respect to the effect(s) of said drugs or combination or panel or group of drugs thereof, data with respect to adverse effects, data with respect to additional clinical parameters, such as patient specific data and/or treatment history, data with respect to the stratification and/or monitoring, and data for automatization of the system, for example for controlling at least one robot.

Item 25. The testing system according to any one of Items 22 to 24, further comprising at least one incubation timer.

Item 26. The testing system according to any one of Items 22 to 25, further comprising at least one drug reservoir, and/or tissue sample dissociation unit.

Item 27. The testing system according to any one of Items 22 to 26, further comprising at least one means for determining physiological parameter selected from size determination of said 3D microtissue, quantification of internal reporter gene expression in said 3D microtissue, determination of the intracellular ATP content in said 3D microtissue, and determination of pre-selected biomarkers in said 3D microtissue, wherein preferably said means for size determination of said 3D microtissue comprise means for at least one parameter selected from diameter, perimeter, volume, and area of optical cross section, preferably imaging devices, and means for the analysis of growth-kinetics.

Item 28. A computer program comprising means for controlling the testing system according to any one of Items 22 to 27, in particular a computer program comprising data for at least one patient-specific drug or combination or panel or group of drugs as identified and/or selected according to a method according to any one of Items 1 to 16, wherein preferably said computer program is implemented on the testing system according to any one of Items 22 to 27.

Item 29. Use of the testing system according to any one of Items 22 to 27 or computer program according to Item 28 for generating at least one patient-specific drug or combination or panel or group of drugs.

Item 30. At least one patient-specific drug or combination or panel or group of drugs as identified and/or selected according to a method according to any one of Items 1to 16 or 19.

The elements of the invention are described herein. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

FIG. 1 shows exemplary relations of in vivo pharmacokinetic and resulting deduced in vitro treatment time in the methods according to the present invention. t½ is drug half-life, tₘₐₓ is the time between the administration of a drug to reaching the maximum plasma concentration (Cₘₐₓ).

FIG. 2 illustrates the general workflow of a preferred embodiment of the method according to the invention, i.e. the exposure-time dependent drug addition and incubation on cells/tissues.

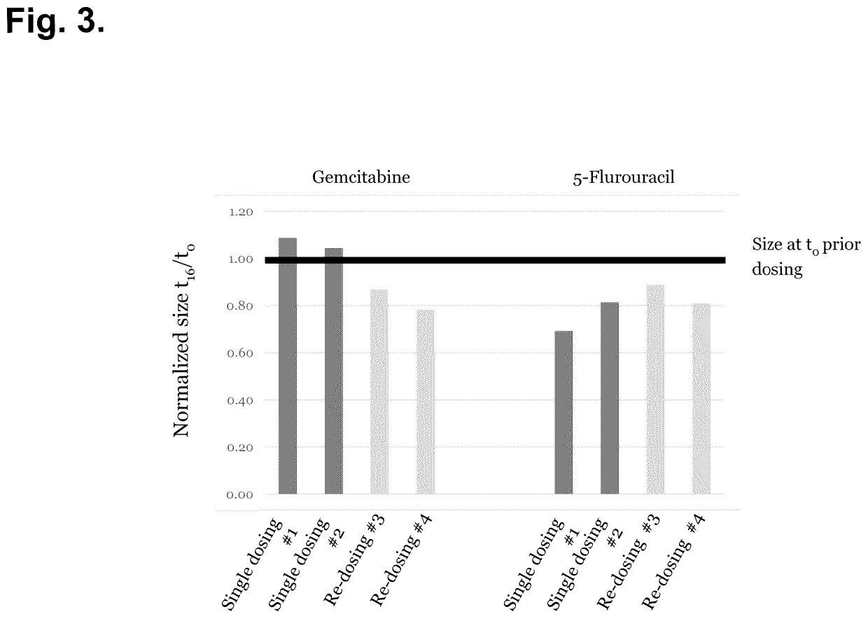

FIG. 3 shows the impact of drug dosing, highlighting the importance to integrate pharmacokinetic parameters and dosing schedules into a-in this case-long-term cell-based drug response test set up. A pancreatic cancer microtumor model consisting of Panc-1 cancer cells and NIH3T3 fibroblasts were treated with either Gemcitabine or 5-Flurouracil. Both drugs were dosed either once for 7 hours or re-dosed for another 7 h with a 48 h recovery time in between (four independent experiments). Whereas the redosing of Gemcitabine was leading to an increased efficiency, 5FU efficiency did not improve.

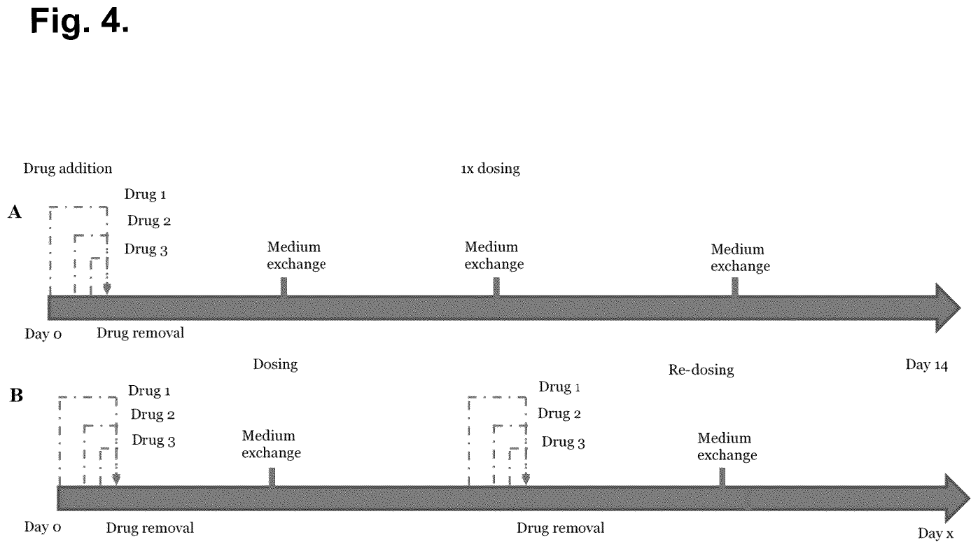

FIG. 4 shows a schematic overview of a drug testing.

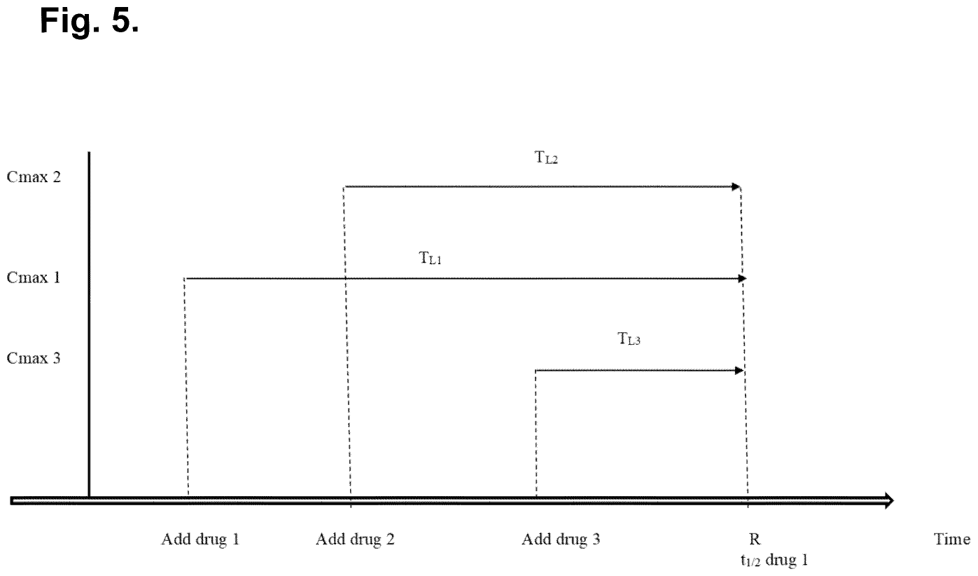

FIG. 5 shows a schematic overview of a drug testing scheme with three different drugs.

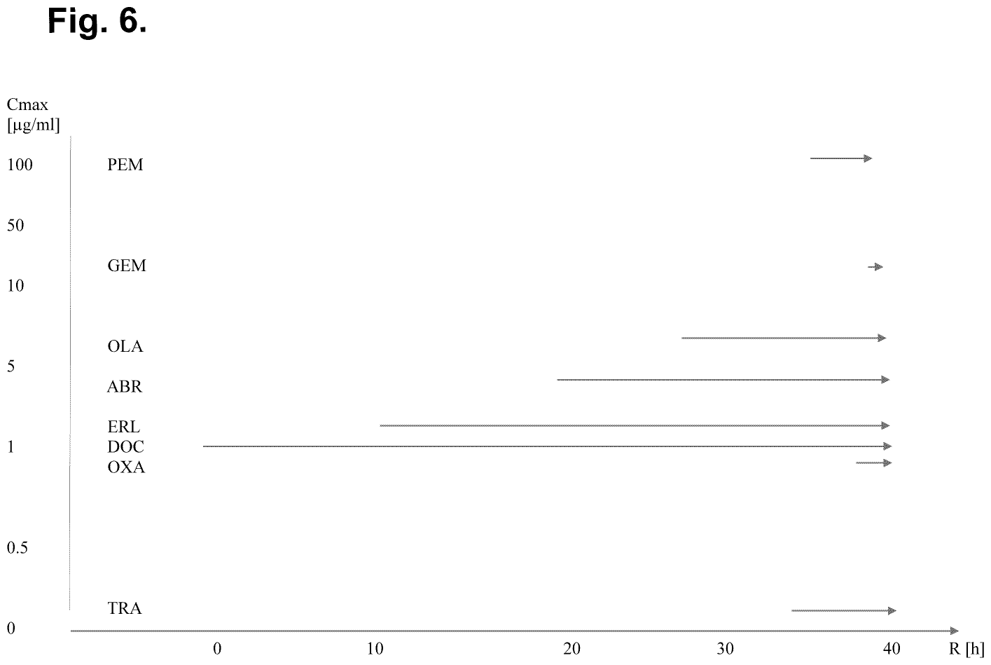

FIG. 6 shows a schematic overview of a drug testing scheme according to table 1 (one round of dosing).

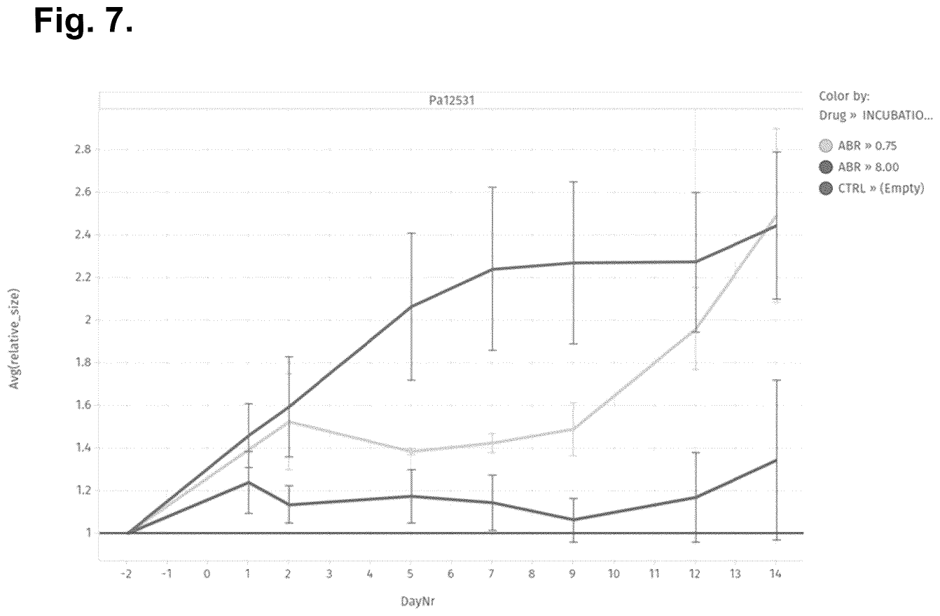

FIG. 7 shows the impact of drug dosing, highlighting the importance to integrate pharmacokinetic parameters and dosing schedules into a—in this case-long—term cell-based drug response test set up. See example “Time dependent dosing example with pancreatic cancer”.

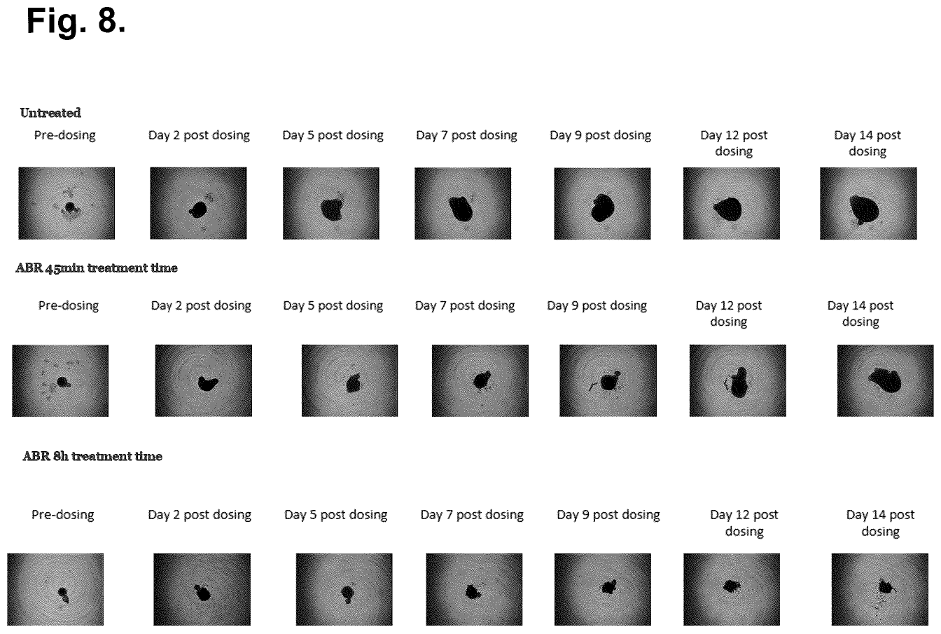

FIG. 8 also shows the impact of drug dosing, highlighting the importance to integrate pharmacokinetic parameters and dosing schedules into a—in this case-long—term cell-based drug response test set up. See example “Time dependent dosing example with pancreatic cancer”.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples. For the purposes of the present invention, all references as cited herein are herewith incorporated by reference in their entireties.

### Tissue Preparation and Microtissue Generation

A tissue biopsy sample obtained from a human cancer patient was placed into an appropriate medium in form of a transport solution comprising antibiotics. Optionally, a hypotonic solution can be added for further processing.

The tissue biopsy sample material was shipped at 4° C. For a 100 mg tissue biopsy sample, a volume of 1.5 mL of transport medium comprising the tissue sample was used.

For haemolysis in order to lyse red blood cells, the tissue sample was then subjected to sonification using a commercially sonificator at 1 MHz, 2 Wcm⁻² for 5 mins. Care was taken to prevent heating of the tissue sample so as to substantially maintain the integrity of the cells, while maintaining the temperature at 4° C. The haemolysis step was performed for 5 mins.

After sonification, the medium was removed and replaced by a medium for tissue dissociation. For this, lytic suitable enzyme(s) (selected from Collagenase, Trypsin, Elastase, Hyaluronidase, Papain, Chymotrypsin, Deoxyribonuclease I, and Neutral Protease (Dispase)) in an appropriate buffer system were used for four cycles, each for 12minutes. The number and length of the cycles may be adjusted to the progress of the tissue digestion, for example by first using harsher and then afterwards exceedingly milder conditions.

The tissue dissociation took place in a volume of 1 ml at a temperature of 37° C. and for 60 minutes.

Then, a stop (STOP) solution (6 ml) containing horse serum and solubilized collagen was added to the lysis solution in order to stop the enzymatic activity.

The final volume of this reaction mixture was 10 ml and the solution was kept at 4° C. The cells as obtained were washed to remove cell debris by gravity enforced sedimentation of cells for 10 minutes in 2×5 ml in production medium (DMEM+10% FCS). Subsequently, any large tissue debris was removed by filtering 5 ml of the solution comprising the cells through a 200 μm pore size filter and further addition of 5 ml production medium.

Thus obtained cells were transferred to a microwell plate, in this case a 384 well plate pre-loaded with production medium and subsequently incubated at a temperature of 37° C. -

For each well, 25 μl of the cell suspension comprising cells were added to 50 μl production medium previously filled into the cavities of the multiwell plate. In addition, 25 μl of a solution comprising supporting cells were added to each well.

In a first embodiment/option, the thus obtained plate was transferred directly into a so-called profiling device (unit) for tissue maturation (microtissue generation).

In a second embodiment/option, a separate tissue maturation was performed within a so-called creator unit. This requires an additional incubator and imaging capacity, but does not impact the throughput of the profiler unit, which can be used for testing of other tissues.

### Identification of Elimination Half-Life (t½) for Drugs to be Tested

The elimination half-life or drug half-life tin generally refers to the time required for half the dose of drug administered to be removed from the body. For those drugs to be tested, where a suitable tin can not be taken from the literature, and/or for the purpose of grouping or forming panels of drugs, tin values may be determined as follows.

In accordance with the existing guidelines, the rate (Cₘₐₓ, tₘₐₓ) and extent (AUC) of absorption of drug from a test formulation (vs. reference formulation) is evaluated using a single dose study in healthy volunteer subjects, followed by measuring the blood/plasma concentration of the parent drug and any major active metabolites (if present) for a time period of ≥3 t½.

tₘₐₓ is usually related to the time period/length of t 1/2, as drugs with short half-lives tend to peak and are eliminated quickly, often requiring more frequent dosing or redosing to maintain a drug within its clinically effective therapeutic range (see also below).

For a determination of the parameters, blood samples are taken at 0, 1, 2, 3, 4, 5, 6, 7, and 8 hours, optionally followed by 10 and 12, 18, 24, 30, 36, and 42 hours after administration. As a general rule, the more frequently samples around the expected time of the maximum concentration are taken, the more accurate the value of t½ (and tₘₐₓ and Cₘₐₓ) are. The above determinations can be made in several individuals, and median values can be determined as parameter e.g. for grouping (see below), and testing.

### Pre-Selection and/or Grouping of the Combination or Panel of Drugs to be Tested

Grouping or pre-selection of drugs can be achieved based on one or more of the following parameters that are known to the person of skill:
i) Cₘₐₓ—the value defining the maximal concentration used,
ii) t½, —time until 1/2 of the drug is cleared by the patient/cells (half-life),
iii) Duration of action [h] (length of time that particular drug is effective),
iv) Elimination time [h] (time it takes for the concentration of the drug in the plasma or the total amount in the body to be removed),
v) Area under the curve (AUC) [ng*h/ml],
vi) Clearance (CL) [L/h] (is equal to the rate at which a drug is removed from plasma (mg/min) divided by the concentration of that drug in the plasma (mg/mL))
vii) Minimal effective concentration (MEC) (minimum plasma concentration of a drug needed to achieve sufficient drug concentration at the receptors to produce the desired pharmacologic response, if drug molecules in plasma are in equilibrium with drug molecules in the various tissues),
viii) Subtherapeutic level (dose of a drug that does not achieve a particular therapeutic effect),
ix) tₘₐₓ, i.e. time until Cₘₐₓ is reached.

As a preferred example, if 2x the half-life is chosen as the main determinant for

the in vitro incubation time, a grouping (here: three groups) of selected drugs results in the following Table 1:

| | | | In vitro | Exposure |
|---|---|---|---|---|
| | | | exposure | time |
| INN (Name) | t_{1/2} [h] | 2 × t_{1/2} [h] | time [h] | Group |
| Erlotinib | 24.4 | 48.8 | 65 | 1 |
| Docetaxel | 41 | 82 | 65 | 1 |
| Trametinib | 4.8 | 9.6 | 14 | 2 |
| Olaparib | 11.9 | 23.8 | 14 | 2 |
| Pemetrexed | 4.4 | 8.8 | 14 | 2 |
| Fluorouracil | 0.3 | 0.6 | 0.6 | 3 |
| Gemcitabine | 0.23 | 0.46 | 0.6 | 3 |

The drugs are then applied/distributed based on the pre-made drug panel in the order of their exposure time group to allow for sequential incubation times. The grouped drugs can be composed of exploratory and/or approved drugs. Preferably, the grouping is performed using a drug properties' database and a computer program for generating the panels according to the above parameters. Preferred is the parameter t½ or 2x t½.

### First Testing of Drugs

For the characterization (profiling) of the cultured cells in the presence of a drug or combinations of drugs, a 384-well plate was pre-loaded with maturated microtissues (if not maturated in the device, as mentioned above).

The loaded plate was moved to an incubator using an automated, robotic device, such as a KUKA LBR Med lightweight robot. The plate was placed into the incubator, and was further moved to an imaging unit. The 384-well plate was then image-analyzed in a reader, for example, a QC imager for 10 minutes. The plate was then moved to a liquid handling unit/station, and the growth medium was exchanged.

Then, the first drug, group (combination) of drugs or panel of drugs to be tested was contacted with the microtissues. About 30 mL were necessary per 384-well plate. Drugs were supplied in form of a pre-fabricated drug solution matrix in medium on a separate 384-well plate, and administered (each) at Cₘₐₓ in accordance with the present invention. The operation was performed by a robot controlled by a respective software.

As an option, the plate was then moved again to the imaging system and subjected to imaging in a 384-well plate HD-imaging system for between 10 and 30 minutes (i.e. 10, 15, 20, 25 or 30 minutes, depending on the t½ of the first drug, group (combination) of drugs or panel of drugs to be tested. The plate was then moved back to the incubator unit, and the microtissue cells were incubated for (in this case) 2 hours, which equaled R/2

Then, the second drug, group (combination) of drugs or panel of drugs to be tested was contacted with the microtissues. Again, about 30 mL were necessary per 384-well plate. Drugs were supplied in form of a pre-fabricated drug solution matrix in medium on a separate 384-well plate, and administered each at Cₘₐₓ in accordance with the present invention. The operation was performed by a robot controlled by a respective software.

Again as an option, the plate was then moved to the imaging system and subjected to imaging in a 384-well plate HD-imaging system for between 10 and 30 minutes (i.e. 10, 15, 20, 25 or 30 minutes, depending on the tin of the second drug, group (combination) of drugs or panel of drugs to be tested. The plate was then moved back to the incubator unit, and the microtissue cells were incubated for (in this case) 2 hours, reaching time point R.

The drugs as tested are then removed and the plate is then moved from the incubator unit to a liquid handling station. The medium was exchanged two times in a period of about 20 minutes, and medium without drugs is added.

As an option, the plate is moved to the imaging system and subjected to QC-imaging for 10 minutes. Thereafter, the plate is moved again to the incubator unit and is incubated until 3.5 days are reached. The medium was then exchanged for two more times, at day 7 and 10.5.

In an embodiment of the testing protocol, either a (at least one) re-dosing step (e.g. at day 7) using the same drug, group (combination) of drugs or panel of drugs to be tested as above, or a (at least one) different dosing step using a different drug, group (combination) of drugs or panel of drugs to be tested, or a mix of the same or different substances can be introduced.

On the last day of the test, here day 14, the microtissue(s) is/are subjected to analysis. The plate is removed from the incubator unit, and it is moved to the imaging unit, where it is subjected to HD imaging. As optical readout options, the size may be taken as the primary readout and multiple parameters may be selected as secondary options, such as at least one parameter selected from diameter, perimeter, volume, and area of optical cross section.

The respective data was collected, and stored for further analysis. All steps were performed by a robot controlled by a respective software.

### Additional Testing Layouts with Different Pharmacokinetics

In another embodiment, a first test-drug having a t½ of 6 hours is added to a microtissue-culture at a predetermined concentration (Cₘₐₓ) on day 0 of the assay. A second test-drug having a t½ of 3 hours is added 3 hours later, again at a predetermined concentration (Cₘₐₓ). A third test-drug having a t½ of 1.5 hours is added 1.5 hours later again at a predetermined concentration (Cₘₐₓ). After a joint incubation of 1.5 hours, all drugs as tested consequently reach their t½ at the same time (R).

Testing conditions and analysis were in accordance with the above, except there was no re-dosing step.

In yet another embodiment, a first test-drug having a t½ of 8 hours is added to a microtissue-culture at a predetermined concentration (Cₘₐₓ) on day 0 of the assay. A second test-drug having a t½ of 4 hours is added 4 hours later, again at a predetermined concentration (Cₘₐₓ). A third test-drug having a t½ of 1.5 hours is added 2.5 hours later again at a predetermined concentration (Cₘₐₓ). After a joint incubation of 1.5 hours, all drugs as tested consequently reach their t½ at the same time (R).

In a re-dosing step the first test-drug having a t½ of 8 hours is added again to the microtissue-culture at a predetermined concentration (Cₘₐₓ) on day 0 of the assay. A fourth test-drug having a t½ of 6 hours is added 2 hours later, again at a predetermined concentration (Cₘₐₓ). The third test-drug having a t½ of 1.5 hours is added 2.5 hours later again at a predetermined concentration (Cₘₐₓ). After a joint incubation of 1.5 hours, all drugs as tested consequently reach their t½ at the same time (R).

Testing conditions and analysis were in accordance with the above.

### Redosing Example with Pancreatic Cancer I

A pancreatic cancer microtumor model consisting of Panc-1 cancer cells and NIH3T3 fibroblasts were treated with either Gemcitabine (GEM) and/or 5-Flurouracil (5-FU). Both drugs were dosed either once for 7 hours or re-dosed for another 7 h with a 48 h recovery time in between (four independent experiments).

Whereas the redosing of Gemcitabine was leading to an increased efficiency, 5-FU efficiency did not improve (FIG. 2).

### Example with Several Drugs

For this experiment, a panel of drugs was pre-selected as shown in Table 2, below. The treatment scheme is shown in FIG. 6.

TABLE 2
Panel of drugs as used for the present examples
with parameters as identified. Treatment time =
R = tₘₐₓ + t_{1/2}

| | Cmax | | | Treatment |
|---|---|---|---|---|
| | Human | tₘₐₓ | t_{1/2} | time [h], |
| Compound | [ug/ml] | [h] | [h] | eq. R |
| Gemcitabine (GEM) | 18.5 | — | 0.23 | 0.23 |
| Abraxame (protein- | 4.2 | — | 20.2 | 20.2 |
| bound paclitaxel) | | | | |
| (ABR) | | | | |
| Trametinib (TRA) | 0.022 | 2 | 4.35 | 6.35 |
| Olaparib (OLA) | 5.8 | 1.3 | 11.9 | 13.2 |
| Oxaliplatin (OXA) | 1.2 | — | 1.86 | 1.86 |
| Erlotinib (ERL) | 2.3 | 5.5 | 24.4 | 29.9 |
| Docetaxel (DOC) | 1.5-2.5 | — | 41 | 41 |
| Pemetrexed (PEM) | 90-150 | — | 4.4 | 4.4 |

### Time Dependent Dosing Example with Pancreatic Cancer

Microtumors were produced from pancreatic cancer PDX tissue. Briefly, pancreatic tumor tissue was excised from mice. The tumor tissue was rinsed and minced into small pieces. The tumor pieces were dissociated with an enzymatic solution into single cells. The cells were seeded into a non-adhesive U-bottom plate to self-assemble into microtumors of around 250 μm in diameter. After a maturation phase of 5 days the individual microtumors were dosed with Abraxane, albumin-bound paclitaxel, for 45 min and 8 h respectively. Growth of the tissues were monitored over 14 days by bright-field imaging to monitor not only short-term response but more importantly long-term sustainability. Although both treatment times with Abraxane did result in an initial response pancreatic microtumors treated for only 45 min with Abraxane were re-entering growth after 7 days in contrast to the microtumors treated for 8 h (see FIGS. 7 amd 8).

1. Method for determining the effectivity of at least one, drug or drug combination for the treatment of a patient, said method comprising the following steps:
providing at least one culture of a 3D microtissue comprising a microtumor based on dissociated cells of a tissue sample derived from a patient;
providing at least one drug or a combination of drugs or a panel of drugs to be tested;
identifying a t½ period for each of said drug or combination of drugs or panel of drugs of drugs, wherein the combination of drugs or panel of drugs is selected according to the t½ time periods;
contacting the at least one 3D microtissue culture with the drug or combination of drugs or panel of drugs identified to have a longest t½ time period(s) (TL1), wherein an end of the longest t½ time period defines the time point for a removal R of the drug, the combination of drugs, or the panel of drugs from the culture;
incubating said at least one 3D microtissue culture with the drug or combination of drugs or panel of drugs with the longest t½ time period;
determining an effect of said drug or combination of drugs or panel of drugs thereof on at least one physiological parameter of the at least one 3D microtissue culture;
contacting the at least one 3D microtissue culture with another of the drug or combination of drugs or panel of drugs identified to have a shorter t½ time period compared to the drug or combination of drugs or panel or group of drugs with the longest t½ time period, at one or more time points prior to expiration of timepoint R;
incubating the at least one 3D microtissue culture with the other drug drugs or combination of drugs or panel of drugs with the shorter t½ time period;
removing the other drug or combination of drugs or panel of drugs with the shorter t½ time period from the culture at timepoint R;
determining an effect of the other drug or combination of drugs or panel of drugs with the shorter time periods on the at least one 3D microtissue g culture; and
selecting at least one of the drug or combination of drugs or panel of drugs with the longest t½ time period or other drug or combination of drugs or panel of drugs with shorter t½ time periods or a combination of one or more of the drugs or combination of drugs or panel of drugs with the longest t½ time period and the other drug or combination of drugs or panel of drugs with shorter t½ time periods based on the determined effect as specific to the patient.

2. The method according to claim 1, wherein R a time to tₘₐₓ for the drug or combination of drugs or panel of drugs, wherein R=tₘₐₓ+t½.

3. The method according to claim 1, wherein the at least one culture of the 3D microtissue is contacted with the drug or combination of drugs or panel of drugs at at a maximum Cmax concentration.

4. The method according to claim 1, wherein identifying the t½ value(s) for the drug or combination of drugs or panel of drugs to be tested comprises testing the drug in a subject or a group of subjects, or identifying said t½ from [the] literature or a database.

5. (canceled)

6. The method according to claim 1, wherein said contacting of the at least one 3D microtissue culture with another of the drug or combination of drugs or panel of drugs identified to have a shorter t½ time period comprises an exposure to one drug, or simultaneously to at least two drugs and/or combinations.

7. The method according to claim 1, wherein the incubation time is between 7 to 28 days.

8. The method according to claim 1, wherein each one of the drug or combination of drugs or panel of drugs and each one of the other drug or combination of drugs or panel of drugs are selected from anti-cancer drugs, comprising one or more of alkylating agents, antimetabolites, natural products, hormones, tyrosine inhibitors, chemotherapeutic compounds, anti-cancer antbodies, in particular Gemcitabine, Abraxame, Trametinib, 43 Olaparib, Oxaliplatin, Erlotinib, Erlotinib, 5-FU, Docetaxel, and Pemetrexed.

9. (canceled)

10. The method accordng to claim 1, wherein the patient suffers from, or is being diagnosed for, a neoplastic disease or tumor.

11. The method according to claim 1, wherein said tissue sample is selected from a sub-sample derived from at least one of a primary tissue sample, a primary tumor sample, and a metastasis sample, and wherein said tissue sample has been obtained by a method comprising one or more of core biopsy, tumor resection, liquid biopsy and/or needle aspiration, and/or wherein said tissue sample is frozen and re-thawed prior to generation of said 3D microtissues.

12. The method according to claim 1, wherein the tissue sample is derived by physically dissecting said tissue sample into smaller pieces comprising cells, by treating said tissue sample with a solution comprising at least one enzyme capable of dissociating cells in said tissue sample, wherein the enzyme is selected from a protease, a collagenase, trypsin, elastase, hyaluronidase, papain, chymotrypsin, deoxyribonuclease I, and neutral protease (dispase).

13. The method according to claim 12, wherein said tissue sample is sonicated with ultrasound prior to treating the tissue sample with the solution.

14. The method according to claim 1, wherein step a) comprises further comprising:
adding or removing stroma cells, stromal fibroblasts, endothelial cells and immune cells to said dissociated cells, wherein for each 3D microtissue a predetermined number of cells is provided.

15. The method according to claim 1, wherein said physiological parameter selected from is based on size determination of said 3D microtissue, quantification of internal reporter gene expression in said 3D microtissue, determination of intracellular ATP content in said 3D microtissue, and determination of pre-selected biomarkers in said 3D microtissue, and further wherein said size determination of said 3D microtissue comprises at least one parameter selected from diameter, perimeter, volume, and area of optical cross section.

16. The method according to claim 1 , wherein a fraction of the cells is analyzed, and further comprising:
providing a primary tissue , sample as the tissue sample;
obtaining a subsample in addition to the tissue sample; and
subjecting said subsample to at least one of molecular profiling, histological analysis, and histochemical analysis.

17. A method according to claim 1, comprising:
testing and analyzing said drug or combination of drugs or panel of drugs for adverse effects in said patient.

18. A method to claim 1, comprising:
7 stratifying of said patient based on the patient-specific drug or combination of drugs or panel of drugs or the other drug or combination of drugs or panel of drugs.

19. A method according to claim 1, comprising at least one of:
treating said patient with said at least one or more of the drug or combination of drugs or panel or other drug or combination of drugs or panel of drugs;
adjusting the treatment.

20. (canceled)

21. The method according to claim 1, wherein said method is performed via at least one robot and/or computing apparatus.

22. The method according to claim 1, wherein the method is performed via a testing system, wherein the testing system comprises a unit for culturing an array of the 3D microtissues based on dissociated cells of a tissue sample derived from the patient, a drug testing and another drug testing unit for removing the drug or panel of drugs or combination of drugs to be tested at timepoint R.

23. (canceled)

24. The method according to claim 1, further comprising:
storing data comprising the t½ time periods, data with respect to pre-selection or grouping of the combination or panel of drugs, physiological parameters, data with respect to the effect(s) of said drugs or combination of drugs or panel of drugs thereof, data with respect to adverse effects, data with respect to additional clinical parameters data with respect to stratification and/or or monitoring, and data for automatization of at least one robot.

30. (canceled)
